(19) United States
(12) Patent Application Publication
(10) Pub. No.: US 2022/0079520 A1
(43) Pub. Date: Mar. 17, 2022

(54) SYSTEM, METHOD, COMPUTER-ACCESSIBLE MEDIUM AND APPARATUS FOR NEAR INFRARED OPTICAL NEURAL ACCESS WITH HIGH SPATIOTEMPORAL RESOLUTION FOR BRAIN COMPUTER INTERFACES

(71) Applicant: The Trustees of Columbia University in the City of New York, New York, NY (US)

(72) Inventors: KENNETH L. SHEPARD, Ossining, NY (US); SAJJAD MOAZENI, New York, NY (US); KEVIN RENEHAN, New York, NY (US)

(21) Appl. No.: 17/536,630

(22) Filed: Nov. 29, 2021

Related U.S. Application Data
(63) Continuation of application No. PCT/US2020/034682, filed on May 27, 2020.
(60) Provisional application No. 62/853,226, filed on May 28, 2019.

(51) Int. Cl. A61B 5/00

(52) U.S. Cl. CPC A61B 5/6803; A61B 2562/046; A61B 2562/164; A61B 5/0073

(57) ABSTRACT

An exemplary diffuse optical tomography (DOT) device, can be provided, which can include, for example, a flexible substrate, an optical source(s) configured to generate a plurality of near infrared (NIR) photons disposed on the flexible substrate, and a plurality of detectors disposed on the flexible substrate, wherein each of the detectors can be configured to detect a plurality of backscattered NIR photons from an anatomical structure(s) that can be based on the NIR photons. The flexible substrate can be configured to be applied to an anatomical structure. The detectors can be an array of single photon avalanche diode (SPAD) detectors. The detectors can be configured to measure an arrival time of the backscattered NIR photons. The array can be disposed on a CMOS integrated circuit chip, which can be disposed on the flexible substrate.

### CROSS-REFERENCE TO RELATED APPLICATIONS

This application relates to and claims priority from U.S. Patent Application No. 62/853,226, filed on May 28, 2010, the entire disclosure of which is incorporated herein by reference.

### STATEMENT REGARDING FEDERALLY SPONSORED RESEARCH

This invention was made with government support under Contract No. N66001-19-C-4020, awarded by the Defense Advanced Research Projects Agency. The government has certain rights in the invention.

### FIELD OF THE DISCLOSURE

The present disclosure relates generally to neural access, and more specifically, to exemplary embodiments of exemplary system, method, computer-accessible medium, and apparatus for near infrared optical neural access with high spatiotemporal resolution for brain computer interfaces.

### BACKGROUND INFORMATION

Cortex-wide functional brain imaging and assessment of neural activity can be used to facilitate a better understanding of the human brain, and for deciphering neural circuits by visualizing the flow of information across cortical areas. This type of imaging can be beneficial to discover new methods, and provide novel medical devices to treat and cure neurodegenerative diseases and disorders. Additionally, the computing and sensing paradigms can be shifted toward more efficient bio-inspired procedures by enhancing understanding of the computation mechanisms present in brain dynamics. Finally, functional brain imaging technologies are facilitators of high-bandwidth brain-computer interfaces (“BCI”). Such functional imaging technologies can achieve single-neuron spatial resolution (e.g., sub-50 um), single action potential temporal precisions (e.g., milliseconds) over large areas of cortex (e.g., more than 1M neurons) while being non-invasive.

Implanted electrode arrays for electrophysiological neural activity recording have provided high-temporal resolution, although over a very limited number of neurons (e.g., state-of-the-art 384 recording electrodes per shank. (See, e.g., Reference 1). With the advent of genetically encoded Calcium/voltage indicators (“GEC/VI”), advanced optical fluorescence microscopy methods such as 2-photon imaging have been proposed to image large areas of brain cortex (e.g., mm-by-mm area) without the loss of temporal precision. However, the scattering and absorption produced by the scalp, skull, and brain tissue requires surgical processes to open the skull for these procedures.

Additionally, all other non-invasive imaging procedures either suffer from poor spatial or temporal resolution. Electroencephalogram (“EGG”) electrode arrays placed over the scalp provide 10 s-ms timing resolution over large areas of brain with poor spatial resolution, typically in the centimeter range. Functional magnetic resonance imaging (“fMRI”), positron emission tomography (“PET”), and functional near infrared spectroscopy (“fNIRS”) provide relatively high spatial data with sub-mm resolution. (See, e.g., References 2 and 3). However, these methods are based on hemodynamics detection (e.g., correlated with regional neural activity) leading to poor temporal resolution of seconds (See, e.g., Reference 4).

Thus, it may be beneficial to provide an exemplary system, method, computer-accessible medium, and apparatus for near infrared optical neural access with high spatiotemporal resolution for brain computer interfaces which can overcome at least some of the deficiencies described herein above.

### SUMMARY OF EXEMPLARY EMBODIMENTS

An exemplary diffuse optical tomography (“DOT”) device, can be provided, which can include, for example, a flexible substrate, an optical source(s) that is configured to generate a plurality of near infrared (“NIR”) photons disposed on the flexible substrate, and a plurality of detectors disposed on the flexible substrate, where each of the detectors can be configured to detect a plurality of backscattered NIR photons from an anatomical structure(s) that can be based on the NIR photons. The flexible substrate can be configured to be applied to an anatomical structure. The flexible substrate can be mounted in a headgear configured to be placed on a head of a person(s). The detectors can be an array of single photon avalanche diode (“SPAD”) detectors. The SPAD detectors can be configured to be active only for a tunable time window. The detectors can be configured to measure an arrival time of the backscattered NIR photons. The array can be disposed on a CMOS integrated circuit chip, which can be disposed on the flexible substrate.

In some exemplary embodiments of the present disclosure, the optical source(s) can include an optical source driver(S), where the optical source(s) and the optical source driver(s) can be disposed on the CMOS integrated circuit chip. The optical source(s) can include an optical source emitter(s), where the optical source emitter(s) can be disposed on the CMOS integrated circuit chip. The optical source emitter(s) can be a vertical-cavity surface-emitting laser (“VCSEL”). The VCSEL can be configured to generate the NIR photons at a wavelength of about 670 nm, about 800 nm, or about 850 nm. Each of the SPAD detectors can include a well implant(s) that can be sized to reduce a probability of carrier generation outside of a multiplication region. The exemplary size of the well implant(s) can be about 3 um to 8 um deep.

The flexible substrate can be a flexible polymide substrate. A plurality of active quenching circuits (“AQC”) can be included, where each of the SPAD detectors can have an AQC coupled thereto. Each of the AQCs can be configured to control a tunable time activation window of its respective SPAD detector. Each of the active quenching circuits is enabled using a programmable delay line. A Time-To-Digital Conversion core can be included, which can be configured to generate a clock signal, where a delay of the programmable delay line can be tuned by a phase of the clock signal. Each of the SPAD detectors can have a region(s) that can surround a multiplication region that can be shielded by metal.

Additionally, an exemplary system, method and computer-accessible medium for determining information regarding an anatomical structure(s) using diffuse optical tomography (“DOT”), can include, for example controlling a source to generate a plurality of near infrared (“NIR”) photons to provide to the anatomical structure(s), receiving signals from a plurality detectors indicative of a plurality of backscattered NIR photons received from the anatomical structure(s) that can be based on the NIR photons, determining a time of flight for each of the received backscattered NIR photons based on the signals, rejecting a portion of the signals based on a time of flight of the received backscattered NIR photons, determining an absorption of the NIR photons by the anatomical structure(s) only based on non-rejected signals, and determining the information regarding the anatomical structure(s) based on the absorption.

In some exemplary embodiments of the present disclosure, the source can be controlled to generate the NIR photons at a wavelength of about 670 nm, 800 nm, or about 850 nm. The anatomical structure(s) can be a head of a person. The detectors can be single photon avalanche diode detectors. Each of the detectors can be activated for a tunable time window.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is an exemplary illustration of an exemplary use of the exemplary NIR ToFF-DOT system according to an exemplary embodiment of the present disclosure;

FIG. 1B is an exemplary diagram of the exemplary NIR ToFF-DOT system according to an exemplary embodiment of the present disclosure;

FIG. 2 is an exemplary schematic diagram of the exemplary NIR ToFF-DOT system according to an exemplary embodiment of the present disclosure;

FIG. 3A is an exemplary schematic diagram of a Hybrid TDC according to an exemplary embodiment of the present disclosure;

FIG. 3B is an exemplary transistor-level schematic diagram of an inverter unit buffer cell according to an exemplary embodiment of the present disclosure;

FIG. 4 is an exemplary diagram illustrating a low-jitter SPAD according to an exemplary embodiment of the present disclosure.

FIG. 5A is an exemplary micrograph of a single-pixel ASIC chip according to an exemplary embodiment of the present disclosure;

FIG. 5B is an exemplary exploded-view micrograph of a 3×6 monolithic SPAD array for a photo detection according to an exemplary embodiment of the present disclosure;

FIG. 6 is a flow diagram of an exemplary method for determining information regarding an anatomical structure using diffuse optical tomography DOT according to an exemplary embodiment of the present disclosure; and

FIG. 7 is an illustration of an exemplary block diagram of an exemplary system in accordance with certain exemplary embodiments of the present disclosure.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The exemplary system, method, computer-accessible medium, and apparatus, according to an exemplary embodiment of the present disclosure, can be provided which can be used as/for BCI, brain functional imaging, breast cancer detection, and stroke detection, although not limited thereto.

The exemplary system, method, computer-accessible medium, and apparatus, according to an exemplary embodiment of the present disclosure, can utilize enhanced diffuse optical tomography (“DOT”) using near infrared (“NIR”) light. NIR DOT has been demonstrated previously (see, e.g., Reference 5) achieving millimeter spatial resolution with second-range temporal resolution. The exemplary system, method, computer-accessible medium, and apparatus, according to an exemplary embodiment of the present disclosure, can achieve high spatiotemporal resolution of sub-mm and milliseconds implemented on an integrated wireless device form factor.

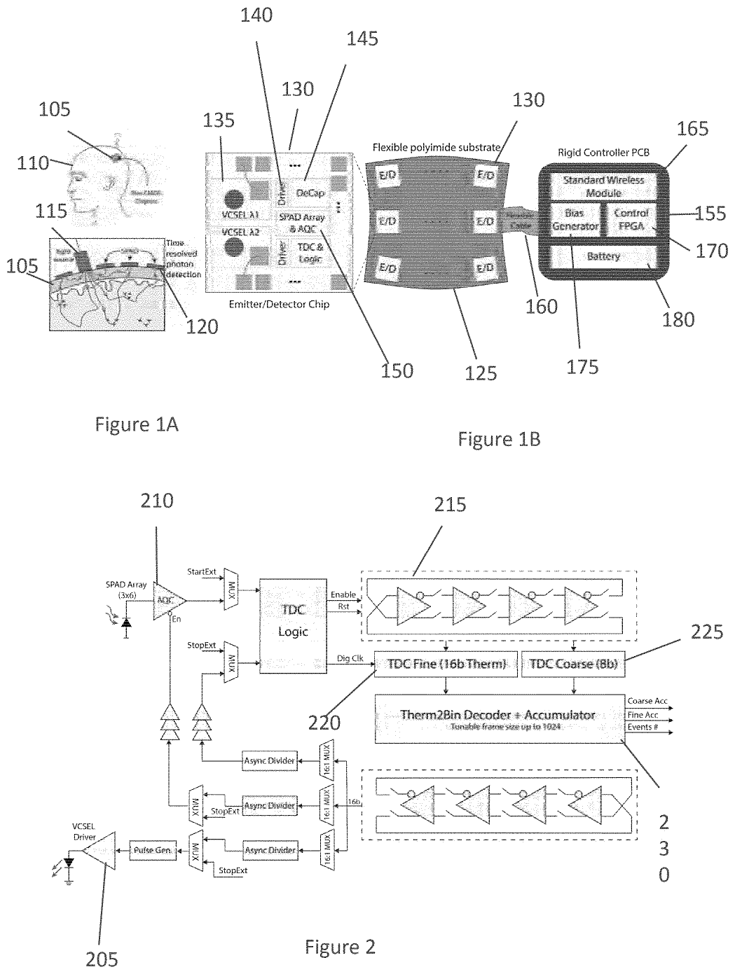

To achieve high spatial and temporal measurements of neural activity from outside the skull, the exemplary system, method, computer-accessible medium, and apparatus, according to an exemplary embodiment of the present disclosure, can utilize diffuse optical tomography combined with time-of-flight and voltage/calcium dependent indicators of neural activity. This new “Time-of-Flight enhanced Functional Diffuse Optical Tomography” (“ToFF-DOT”) can benefit from advances in computational imaging in scattering media, in single-photon detectors with picosecond temporal resolution, and in high-throughput methods to create genetically encoded indicators of neural activity. (See, e.g., FIG. 1A).

In particular, FIG. 1A shows an exemplary illustration of an exemplary use of an exemplary NIR ToFF-DOT system 105 according to an exemplary embodiment of the present disclosure. As shown in FIG. 1A, the exemplary NIR ToFF-DOT system 105 can be placed on the head 110 of a person. The exemplary NIR ToFF-DOT system 105 can include one or more light sources 115 (e.g., laser(s), visible light source(s), NIR source(s), etc.), and a plurality of single photon avalanche diode (“SPAD”)-based detectors 120.

The exemplary system, method, and computer-accessible medium, according to an exemplary embodiment of the present disclosure, can utilize a wearable NIR imaging array 125 in a flexible form factor, as shown in FIG. 1B. In particular, FIG. 1B shows an exemplary diagram of the exemplary NIR ToFF-DOT system according to an exemplary embodiment of the present disclosure. The form factor of the exemplary array 125 can be on the scale of about 10-cm-by-10-cm (e.g., plus or minus 10%) with wireless data telemetry. The exemplary array 125 can include, e.g., a plurality of emitters/detectors 130. Each emitter/detector 130 can integrate one or more vertical cavity surface emitting lasers (“VCSELs”) 135 and drive them using a driver 140 and a passive decoupling capacitor (“DeCap”) 145, synchronized with time-gated detectors. This exemplary design can utilize a custom application-specific integrated circuit (“ASIC”) which can be arrayed on a custom flexible printed circuit board. The approximately 30-ps temporal resolution provided by the SPAD-based detectors 120 and SPAD array 150, can facilitate spatial resolution of about 1 mm (e.g., plus or minus 10%). State-of-the-art DOT can exploit the fact that the concentration and localization of photons within scattering tissue varies depending upon the location of the source, and the position of the detector. An array of sources and detectors distributed over a 10 cm by 10 cm region can be used to collect 5 dimensional light transport data (e.g., 2 dimensions for input light source location, 2 dimensions for detector location and 1 dimension for time of flight). The optical imaging data can serve as an input to a partial differential equation (“PDE”)-constrained image reconstruction procedure, which makes use of the equation of radiative transfer (“ERT”).

As shown in FIG. 1B, the exemplary array 125 can be composed of and/or include a flexible polymide substrate, and can be connected a rigid controller printed circuit board (“PCB”) 155 using a flexible cable 160. Rigid controller PCB can include a standard wireless module 165 for communicating information generated using exemplary array 125. A control field programmable gate array (“FPGA”) 170 can be used to control exemplary array 125. Rigid controller PCB 165 can also or alternatively include a bias generator 175 and a battery 180.

The exemplary system, method, computer-accessible medium, and apparatus, according to an exemplary embodiment of the present disclosure, can achieve mm-scale with millisecond temporal resolution that can be utilized for noninvasive brain functional imaging and BCI devices.

### Exemplary ToFF-DOT Millisecond Temporal Resolution

While optical tomography can currently measure neural activity based on hemodynamics, the temporal resolution can be limited by a physiological response time of several hundred milliseconds. Indeed, optical readout technologies based on blood flow and/or oxygenation can be too slow for a low latency BCI. Fluorescence indicators of neural activity have millisecond-scale temporal resolution, but the excited state lifetimes of these fluorophores can be several nanoseconds, which can destroy time-of-flight information.

The exemplary system, method, and computer-accessible medium, and apparatus, according to an exemplary embodiment of the present disclosure, can be used to measure neural activity based on changes in absorption rather than changes in fluorescence. Because absorption occurs nearly instantaneously, measurements of absorption can preserve ToF information. Thus, the exemplary ToFF-DOT procedure can dramatically improve the spatial and temporal resolution of non-invasive neural read channels. The exemplary system, method, computer-accessible medium, and apparatus, according to an exemplary embodiment of the present disclosure, can improve the signal to noise ratio using spectral properties of the calcium/voltage-dependent absorbers (“CDAs”/“VDAs”), which can be distinct from endogenous time-varying absorbers like hemoglobin.

For example, a 96-well plate screening platform can be modified to evolve indicators with the highest ratio of absorption between action potential and resting conditions. Indicators with the highest change in chromophore absorption can be evolved. A plurality of NIR absorbing voltage and calcium indicators (see, e.g., References 6 and 7) can be used as base constructs, and those with the greatest change in absorption can be selected for further directed evolution. These base constructs can include opsin-based voltage indicators (e.g., Arch, Ace, and Quasr), fluorescent protein-based indicators (e.g., mRubyVSP and VARNUM), and biliverdin containing NIR protein-based calcium and voltage indicators. Opsin-based voltage indicators show dramatic changes in absorption in the NIR, however, their very high temporal response characteristics can make the signals obtained too short in duration for reliable detection using a time correlated imaging method. Calcium and voltage indicators can be tested with slower response characteristics. These can include biliverdin containing-NIR absorption voltage and calcium indicators. Such indicators can have poor performance as fluorescence emitters due to the relatively low quantum yields (e.g., 0.01-0.02) in this family of proteins. However, for the exemplary system, method, computer-accessible medium, and apparatus, based on changes in absorption, these proteins can be optimal.

### Exemplary ToFF-DOT Spatial Resolution

The time of flight of the photons at each of the detectors can be measured, according to an exemplary embodiment of the present disclosure. This temporal information can be used to reject many of the photons that can be scattered by the skull, and that did not interact with the brain. The exemplary time-gating detection procedure can facilitate the reduction in the emitter and detector distance in each measurement to improve the lateral spatial resolution to sub-mm. In addition, the 30 ps time resolution achieved by the exemplary detectors can facilitate the localization of light delivery and collection from a voxel that can be approximately 1 mm³. By additionally exploiting the sparsity of activation achieved by genetically engineered proteins, this spot can be super-resolved by 10-20× and achieve target effective spatial resolution as high as 50 microns. The block-diagram of each exemplary emitter/detector ASIC chip is shown in FIG. 2.

For example, FIG. 2 illustrates an exemplary schematic diagram of the exemplary NIR ToFF-DOT system according to an exemplary embodiment of the present disclosure. A ring-oscillator can be used to generate a finely delayed set of signals that can be used to drive the VCSEL (e.g., using VCSEL Driver 205) to generate short and fast optical pulses. AQC 210 can be used to activate the exemplary SPAD array to measure the ToF in certain time windows. The event driven TDC 215 can be used to indirectly measure the ToF from the photons' arrival time and pulses driving the VCSEL. TDC coarse values 220 and TDC fine values 225 can be generated by a decoder block, and can be fed in or otherwise provided to the digital back-end 230 for time binning and for providing the data in the central FPGA.

### Exemplary Time-Gating TOF Imaging

A limiting factor in prior DOT imaging systems/methods can be the spacing of the emitters and detectors. The minimum spacing can be conventionally limited to cm-range due to the strong reflections and scattering of the photons in the skull before reaching the brain cortex area. In order to overcome this limit, the exemplary system, method, computer-accessible medium, and apparatus can utilize time-gating TOF imaging. In this exemplary procedure, the SPADs can be only activated for a tunable time window. This can be performed using an active quenching circuit (“AQC”) that can be facilitated by a programmable delay line with the resolution of 30 ps. Fine tuning of the delay can be done by selecting a different phase of the clock generated by a high-resolution hybrid high-resolution hybrid Time-To-Digital Conversion (“TDC”) core. A coarse delay line can be implemented via a chain of clock buffers. In doing so, photon detection can be limited only to photons that have passed through the region of interest in the cortex area based on their arrival time interval.

### Simultaneous Direct Neural Activity and Hemodynamic Imaging

Although the exemplary device relies on direct detection of CDA/VDA absorptions for neural activity recording, the hemodynamics in the brain can modulate the absorption properties of the tissue and can lead to a non-constant background absorption level. By integrating VCSEL sources at two different wavelengths (e.g., about 670 nm, about 800 nm, and about 850 nm, plus or minus about 10%) on each chip, both the direct neural activity and changes due to the hemodynamics in brain tissue can be monitored. Monitoring hemodynamics can be used to improve the exemplary device imaging quality by cancelling the hemodynamic absorption in the background. Additionally, this feature can be utilized to combine both modalities, traditional DOT and the exemplary NIR ToFF-DOT procedure, to validate the exemplary device's functionality and study the correlation between neural activity and hemodynamics.

### Exemplary Hybrid High-Resolution TDC

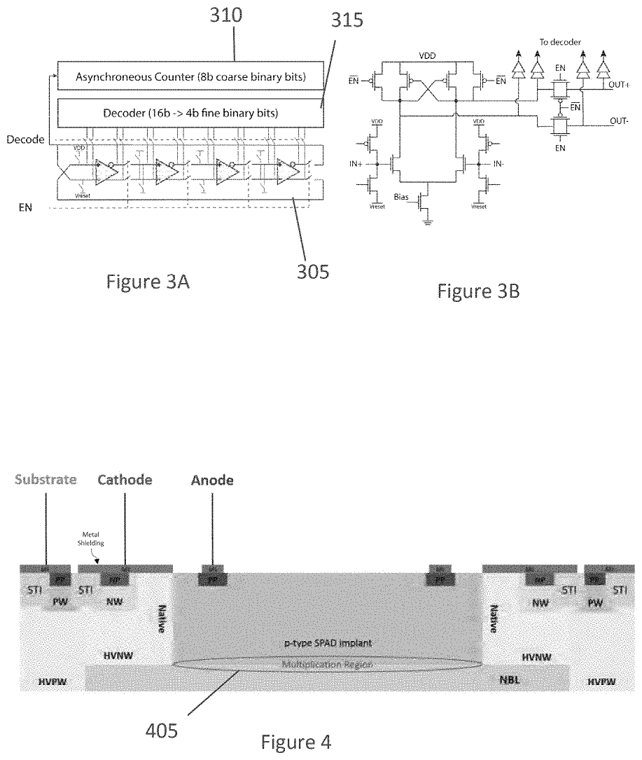

Resolving 1 mm distances in ToF imaging can benefit from 30 ps temporal resolution of the received scattered photons at the detectors. As such, the time-to-digital converter block needs 30 ps or better timing resolution. However, monolithic SPAD arrays with high-efficiencies can be implemented in relatively old CMOS technology nodes where achieving this timing resolution can be challenging. The exemplary system, method, computer-accessible medium, and apparatus can utilize TDC architecture to solve this challenge. FIG. 3A shows an exemplary schematic diagram of a Hybrid TDC 305 according to an exemplary embodiment of the present disclosure. Tins exemplary Hybrid TDC 305 can utilize both active and passive delay elements in a ring-oscillator based design, to achieve 30 ps time delay in-between internal states of the oscillator. This exemplary configuration can eliminate the need to have phase interpolators or time-difference amplifiers, which can utilize extra area-power and calibration. (See, e.g., Reference 11). Additionally, the exemplary Hybrid TDC 305 can be coupled to an asynchronous counter 310 and a decoder 315. The delay inverter buffer cell schematic is shown in the schematic diagram of FIG. 3B. The active inverter can be composed of a differential pair with cross-coupled PMOS loading and PMOS current bleeders to reduce the delay time. (See, e.g., Reference 12). Passive delay elements can be designed via pass-gate switches that can be reused to start and stop the oscillator during the time measurement. Post-layout extracted simulations show approximately 30 ps timing resolution with less than 4.5 mW power per detection with the total compact area of 40 microns by 34 microns.

As shown in the schematic diagram of FIG. 3B, a differential pair with a cross-coupled load with reset mechanism at the input and pass-gate switches at the output can be used to activate and stop the exemplary oscillator. Outputs can be buffered to the decoder block to read-out the phase of oscillator for TDC fine measurement.

### Exemplary Low Jitter Near-Infrared SPAD Design for Time-Gating Fluorescence Imaging

The spatial resolution of a ToF imager can be dependent on how accurately the tuning information associated with rejected photons can be extracted. Uncertainty in the temporal response of a component within the imager can degrade the spatial resolution. In SPADs, this uncertainty arises from the diffusion tail and from jitter. While SPADs have become the preferred optical detector for ToF imaging applications because of their high sensitivity and relatively low jitter, care must be taken in the design of the SPAD to ensure that timing uncertainty can be reduced as much as possible (See, e.g., Reference 8).

Timing uncertainty inherent to the exemplary SPAD structure can be the result of several different effects. The most significant of these effects can be the uncertainty in the region of the exemplary SPAD in which carriers can be generated as a result of an incident photon; carriers generated in the depletion region of the exemplary SPAD can generate an avalanche and corresponding output almost immediately, while carriers generated in the space charge region can take tens of picoseconds to reach the multiplication region before triggering an avalanche depending on transit time dispersion. Even worse can be the case in which a carrier can be generated in neutral regions of the exemplary SPAD: these carriers can diffuse around within the exemplary SPAD for several nanoseconds before reaching the multiplication region and triggering an avalanche. (See, e.g., Reference 9). To reduce the effects of these phenomena, three design changes can be made to the SPAD structure: (i) regions of the SPAD surrounding the multiplication region can be shielded by metal, which can reflect the photons that can lead to photogeneration in areas of the exemplary SPAD outside of the multiplication region; (ii) multiplication region diameter can be made small (e.g., 6-12 um) to keep transit time dispersion small; and (iii) well implants within the exemplary SPAD in which avalanche multiplication does not occur can be sized as small as allowable by the CMOS fabrication process so that probability of carrier generation outside of the multiplication region can be reduced.

The exemplary SPAD d can also be optimized for absorption in the near-infrared region. Typical SPAD designs use shallow well implants that often fail to efficiently collect carriers generated deep in the silicon by longer wavelengths of light. (See, e.g., Reference 10). The exemplary NIR SPADs can utilize deeper well implants to extend the multiplication region further into the substrate so that these carriers can be collected with better efficiency. An exemplary well implant size can be from about 3 um to about 8 um deep (plus or minus about 10%). The area can be circular, rectangular, or square shaped, with a radius of about 4.5 um to about 10 um (plus or minus about 10%).

Higher photon detection efficiency (“PDE”) can help ensure better image quality in situations where low photon counts can be expected. The cross-section of the exemplary SPAD according to an exemplary embodiment of the present disclosure is shown in the diagram of FIG. 4. In particular, FIG. 4 illustrates a cross-section of the exemplary SPAD which has 3 contacts: (i) Anode, (ii) cathode, and (iii) substrate. The active area where photons can be detected, which can be used to trigger the exemplary device, is at the lower region of SPAD implant (e.g., shown by region 405). Native regions can act as a guard ring, and metal shields can be used to improve the SPAD jitter performance by avoiding photon detection by other no-active regions.

The exemplary system, method, computer-accessible medium, and apparatus can utilize integrated ASIC chips that can be fabricated in a 0.13 um CMOS technology. This process can facilitate monolithic design of NIR SPAD devices by adding a custom implant mask. Additionally, all mixed-signal circuitry can be utilized to achieve the beneficial specifications in this platform. VCSELs can be bonded on the chips and driven via a wire-bond.

### Exemplary Single Pixel Emitter/Detector ASIC Chip

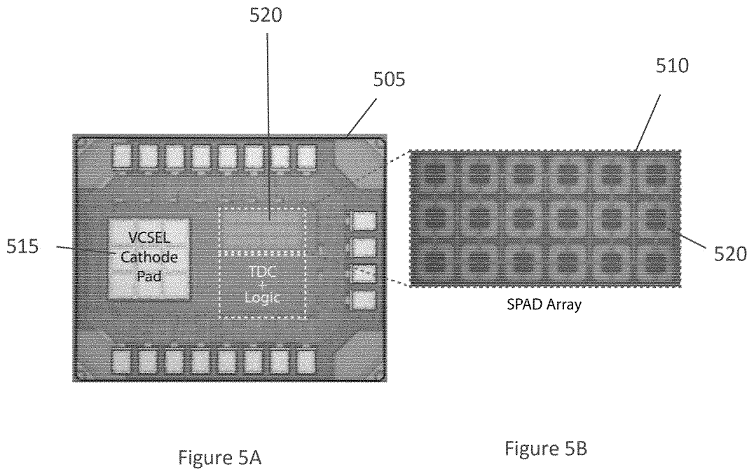

FIG. 5A shows an exemplary micrograph of a single-pixel ASIC chip 505 according to an exemplary embodiment of the present disclosure, and FIG. 5B shows an exemplary exploded-view of a micrograph of a 3×6 monolithic SPAD array 510 for a photo detection according to an exemplary embodiment of the present disclosure. The exemplary custom ASIC can function as both an emitter and detector of near-infrared light. Emitter functionality can be achieved by driving a about 670 nm, about 800 nm, or about 850 nm (plus or minus about 10%) commercial VCSEL 515 that can be directly mounted on ASIC chip 505, while detector functionality can be achieved by monitoring the output of SPADs 520 integrated in CMOS. ASIC chip 505 can act as a single pixel in the completed (or substantially completed) system, and can be the basic functional unit for the ToF imager. The ToF imager can utilize data collected from an array of 400 ASICs to create image frames. Single pixel emitter/detector ASIC chips can be fabricated in TSMC 130 nm BCD process. Customized doping implants can facilitate the integration of NIR SPADs monolithically in this process. Each chip can have a 3×6 array of SPADs to increase the aperture of photon detection. (See e.g., microarray shown in FIG. 5B). All SPADs 520 can be connected together and time-gated via a single AQC. Two VCSELs can be integrated on each chip with cathode and anode connections to the CMOS circuits made via silver paste and wire-bonding, respectively. Each VCSEL can have its own driver circuitry generating short pulses with pulse widths of less than 100 ps.

### Exemplary Flexible PCB Packaged Pixel Array

The entire exemplary device can be fabricated on a 10-cm-by-10-cm flexible PCB, which can be draped over the scalp. The PCB can support a 20-by-20 array of nodes on less than a 5 mm pitch. Each node can be a 1 mm-by-1 mm single-pixel emitter/detector ASIC chip. Discrete electronics on the PCB can facilitate any of the 400 ASICs to act as a source with any of the 400 ASICs acting as a detector. Additionally, a single ASIC can acting simultaneously as source/detector or multiple ASICs operating as source/detector pairs in parallel. A balanced H-tree trace can be used to distribute the synchronous emitters pulse pattern generated by the central FPGA to ASICs with minimal timing skew and jitter. Scan chain signals for configuring ASICs can also be shared to minimize the number of routings on this PCB. Since scan chain signals can be shared, individual enable signals per ASIC chip can be generated via discrete digital decoder chips (e.g., on the flexible PCB), which can be controlled directly by the FPGA.

### Exemplary Central Controller (e.g., Unit)

A conventional PCB integrating commercial block for powering up chips, providing bias signals, controlling and synchronization pixels, collecting image data, and relaying data wirelessly to a computer base station for image processing can be used. Readout electronics on the PCB can facilitate data readout rates of up to, for example, 40 Mb/s via an 802.11 wireless link. A global, digital shutter signal can ensure synchronization of all ASICs during any mode of operation via a Xilinx FPGA (e.g., Spartan series). The FPGA can also set the operation mode by configuring ASICs via separate scan chains. Bias signals including the high-voltage bias for the SPADs, the VCSEL driver's supply, and the TDC current biases can be generated and regulated using discrete components on board the FPGA. For example, a 5 V, 400 mAH battery supply, weighing in at about 10 grams, can support approximately 1 hour of operation.

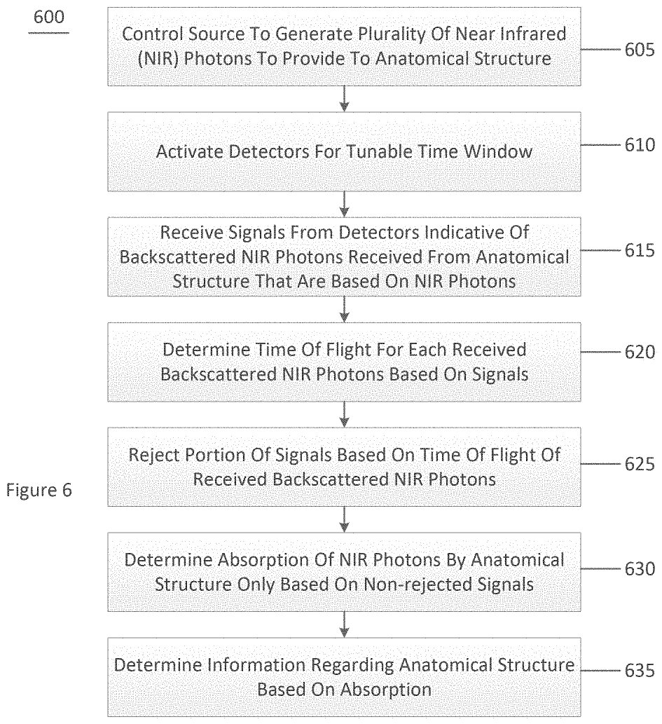

FIG. 6 shows a flow diagram of an exemplary method 600 for determining information regarding an anatomical structure using diffuse optical tomography DOT according to an exemplary embodiment of the present disclosure. In particular, at procedure 605, a source can be controlled to generate near infrared (NIR) photons to provide to an anatomical structure. At procedure 610, detectors can be activated for a tunable time window. At procedure 615, signals from the detectors—indicative of backscattered NIR photons received from the anatomical structure that are based on the NIR photons—can be received. At procedure 620, a time of flight for each of the received backscattered NIR photons can be determined based on the signals. At procedure 625, a portion of the signals can be rejected based on a time of flight of the received backscattered NIR photons. At procedure 630, an absorption of the NIR photons by the anatomical structure can be determined only based on non-rejected signals. At procedure 635, the information regarding the anatomical structure can be determined based on the absorption.

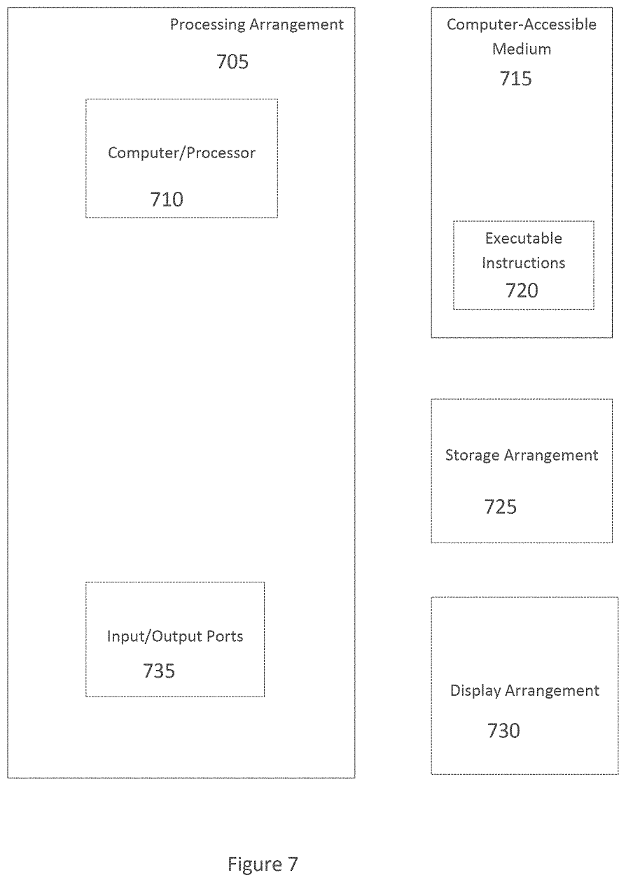

FIG. 7 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 705. Such processing/computing arrangement 705 can be, for example entirely or a part of, or include, but not limited to, a computer/processor 710 that can include, for example one or more microprocessors, and use instructions stored on a computer-accessible medium (e.g., RAM, ROM, hard drive, or other storage device).

As shown in FIG. 7, for example a computer-accessible medium 715 (e.g., as described herein above, a storage device such as a hard disk, floppy disk, memory stick, CD-ROM, RAM, ROM, etc., or a collection thereof) can be provided (e.g., in communication with the processing arrangement 705). The computer-accessible medium 715 can contain executable instructions 720 thereon. In addition or alternatively, a storage arrangement 725 can be provided separately from the computer-accessible medium 715, which can provide the instructions to the processing arrangement 705 to configure the processing arrangement to execute certain exemplary procedures, processes, and methods, as described herein above, for example.

Further, the exemplary processing arrangement 705 can be provided with or include an input/output ports 735, which can include, for example a wired network, a wireless network, the internet, an intranet, a data collection probe, a sensor, etc. As shown in FIG. 7, the exemplary processing arrangement 705 can be in communication with an exemplary display arrangement 730, which, according to certain exemplary embodiments of the present disclosure, can be a touch-screen configured for inputting information to the processing arrangement in addition to outputting information from the processing arrangement, for example. Further, the exemplary display arrangement 730 and/or a storage arrangement 725 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference, in their entireties.
1. Jun, J. J., Steinmetz, N. A., Siegle, J. H., Denman, D. J., Bauza, M., Barbarits, B., . . . Harris, T. D. (2017). Fully integrated silicon probes for high-density recording of neural activity. Nature, 551, 232.
2. Hillman, Elizabeth M C. “Optical brain imaging in vivo: techniques and applications from animal to man” Journal of biomedical optics vol. 12.5 (2007): 051402.
3. Raichle, M. E. (2009). A brief history of human brain mapping. Trends in Neurosciences, 32(2), 118-126. http://doi.org/10.1016/j.tins.2008.11.001
4. Ma, Y., Shark, M. A., Kim, S. H., Kozberg, M. G., Thibodeaux, D. N., Zhao, H. T., Yu, H., . . . Hillman, E. M. (2016). Wide-field optical mapping of neural activity and brain haemodynamics: considerations and novel approaches. Philosophical transactions of the Royal Society of London. Series B, Biological sciences, 371(1705), 20150360.
5. Strangman, Gary, David A. Boas, and Jeffrey P. Sutton. “Non-invasive neuroimaging using near-infrared light.” Biological psychiatry 52.7 (2002): 679-693.
6. Kannan, M., Vasan, G., Huang, C., Haziza, S., Li, J. Z., Inan, H., . . . Pieribone, V. A. (2018). Fast, in vivo voltage imaging using a red fluorescent indicator. Nature Methods, 15(12), 1108-1116.
7. Qian, Y., Piatkevich, K. D., Mc Larney, B., Abdelfattah, A. S., Mehta, S., Murdock, M. H., . . . Campbell, R. E. (2019). A genetically encoded near-infrared fluorescent calcium ion indicator. Nature Methods, 16(2), 171-174.
8. F. Villa, R. Lussana, D. Bronzi, S. Tisa, A. Tosi, F. Zappa, A. D. Mora, D. Contini, D. Durini, S. Weyers, and W. Brockherde, “CMOS Imager With 1024 SPADs and TDCs for Single-Photon Timing and 3-D Time-of-Flight,” IEEE Journal of Selected Topics in Quantum Electronics, vol 20, no. 6, pp. 364-373, 2014.
9. A. Gulinatti, I. Rech, M. Assanelli, M. Ghioni, and S. Cova, “A physically based model for evaluating the photon detection efficiency and the temporal response of SPAD detectors,” Journal of Modern Optics, vol. 58, no. 3-4, pp. 210-224, February 2011.
10. E. A. G. Webster, J. A. Richardson, L. A. Grant, D. Renshaw, and R. K. Henderson, “A Single-Photon Avalanche Diode in 90-nm CMOS Imaging Technology With 44% Photon Detection Efficiency at 690 nm,” IEEE Electron Device Letters, vol. 33, no. 5, pp. 694-696, May 2012.
11. S. Mandai and E. Charbon, “A 128-channel, 9 ps column-parallel two-stage TDC based on time difference amplification for time-resolved imaging,” 2011 Proceedings of the ESSCIRC (ESSCIRC), Helsinki, 2011, pp. 119-122.
12. J. M. Pavia, M. Scandini, S. Lindner, M. Wolf and E. Charbon, “A 1×400 Backside-Illuminated SPAD Sensor With 49.7 ps Resolution, 30 pJ/Sample TDCs Fabricated in 3D CMOS Technology for Near-Infrared Optical Tomography,” in IEEE Journal of Solid-State Circuits, vol. 50, no. 10, pp. 2406-2418, October 2015.

1. A diffuse optical tomography (DOT) device, comprising:
a flexible substrate;
at least one optical source configured to generate a plurality of near infrared (NIR) photons disposed on the flexible substrate; and
a plurality of detectors disposed on the flexible substrate, wherein each of the detectors is configured to detect a plurality of backscattered NIR photons from at least one anatomical structure that is based on the NIR photons.

2. The DOT device of claim 1, wherein the flexible substrate is configured to be applied to an anatomical structure.

3. The DOT device of claim 1, wherein the flexible substrate is mounted in a headgear configured to be placed on a head of at least one person.

4. The DOT device of claim 1, wherein the detectors are an array of single photon avalanche diode (SPAD) detectors.

5. The DOT device of claim 4, wherein the SPAD detectors are configured to be active only during a tunable time window.

6. A DOT device of claim 4, wherein the SPAD detectors are configured to measure an arrival time of the backscattered NIR photons.

7. The DOT device of claim 4, wherein the array is disposed on a CMOS integrated circuit chip, which is disposed on the flexible substrate.

8. The DOT device of claim 7, where the at least one optical source includes at least one optical source driver, and wherein the at least one optical source and the at least one optical source driver are disposed on the CMOS integrated circuit chip.

9. The DOT device of claim 7, where the at least one optical source includes at least one optical source emitter, and wherein the at least one optical source emitter is disposed on the CMOS integrated circuit chip.

10. The DOT device of claim 9, where the at least one optical source emitter is a vertical-cavity surface-emitting laser (VCSEL).

11. The DOT device of claim 10, wherein the VCSEL is configured to generate the NIR photons at a wavelength of about 670 nm, about 800 nm, or about 850 nm.

12. The DOT device of claim 4, wherein each of the SPAD detectors includes at least one well implant that has a particular size to reduce a probability of carrier generation outside of a multiplication region.

13. The DOT device of claim 12, wherein the particular size of the at least one well implant is about 3 um to 8 um deep.

14. The DOT device of claim 4, further comprising a plurality of active quenching circuits, wherein each of the SPAD detectors has an active quenching circuit coupled thereto.

15. The DOT device of claim 14, wherein each of the active quenching circuits is configured to control a tunable time activation window of its respective SPAD detector.

16. The DOT device of claim 14, wherein each of the active quenching circuits is effectuated using a programmable delay line.

17. The DOT device of claim 16, further comprising a Time-To-Digital Conversion core configured to generate a clock signal, wherein a delay of the programmable delay line is tuned by a phase of the clock signal.

18. The DOT device of claim 4, wherein each of the SPAD detectors has at least one region that surrounds a multiplication region that is shielded by metal.

19. The DOT device of claim 1, wherein the flexible substrate is a flexible polymide substrate.

20. A non-transitory computer-accessible medium having stored thereon computer-executable instructions for determining information regarding at least one anatomical structure using diffuse optical tomography (DOT), wherein, when a computer arrangement executes the instructions, the computer arrangement is configured to perform procedures comprising:
controlling a source to generate a plurality of near infrared (NIR) photons to provide to the at least one anatomical structure;
receiving signals from a plurality detectors indicative of a plurality of backscattered NIR photons received from the at least one anatomical structure that are based on the NIR photons;
determining a time of flight for each of the received backscattered NIR photons based on the signals;
rejecting a portion of the signals based on a time of flight of the received backscattered NIR photons;
determining an absorption of the NIR photons by the at least one anatomical structure only based on non-rejected signals; and
determining the information regarding the at least one anatomical structure based on the absorption.

21-24. (canceled)

25. A system for determining information regarding at least one anatomical structure using diffuse optical tomography (DOT), comprising:
a computer hardware arrangement configured to:
control a source to generate a plurality of near infrared (NIR) photons to provide to the at least one anatomical structure;
receive signals from a plurality detectors indicative of a plurality of backscattered NIR photons received from the at least one anatomical structure that are based on the NIR photons;
determine a time of flight for each of the received backscattered NIR photons based on the signals;
reject a portion of the signals based on a time of flight of the received backscattered NIR photons;
determine an absorption of the NIR photons by the at least one anatomical structure only based on non-rejected signals; and
determine the information regarding the at least one anatomical structure based on the absorption.

26-29. (canceled)

30. A method for determining information regarding at least one anatomical structure using diffuse optical tomography (DOT), comprising:
controlling a source to generate a plurality of near infrared (NIR) photons to provide to the at least one anatomical structure;
receiving signals from a plurality detectors indicative of a plurality of backscattered NIR photons received from the at least one anatomical structure that are based on the NIR photons;
determining a time of flight for each of the received backscattered NIR photons based on the signals;
rejecting a portion of the signals based on a time of flight of the received backscattered NIR photons;
determining an absorption of the NIR photons by the at least one anatomical structure only based on non-rejected signals; and
using a computer arrangement, determining the information regarding the at least one anatomical structure based on the absorption.

31-34. (canceled)
